(19) United States
(12) Patent Application Publication
(10) Pub. No.: US 2026/0338904 A1
(43) Pub. Date: Oct. 29, 2026

(54) REAL-TIME RESPIRATION FEEDBACK SYSTEM TO CORRECT RESPIRATION INDUCED FLUCTUATION OF B0 FIELD IN MAGNETIC RESONANCE IMAGING

(71) Applicant: The Board of Trustees of the University of Illinois, Urbana, IL (US)

(72) Inventors: Kwan-Jin JUNG, Champaign, IL (US); Ryan LARSEN, Urbana, IL (US); Yaokun SHI, Urbana, IL (US); Sepideh Sadaghiani FRIBERG, Urbana, IL (US)

(21) Appl. No.: 19/179,144

(22) Filed: Apr. 15, 2025

Related U.S. Application Data
(60) Provisional application No. 63/635,144, filed on Apr. 17, 2024.

(51) Int. Cl. G01R 33/567; A61B 5/00; A61B 5/055; A61B 5/08

(52) U.S. Cl. CPC G01R 33/5673; A61B 5/055; A61B 5/0816; A61B 5/7285

(57) ABSTRACT

Methods, apparatus, and storage medium for compensating respiration induced fluctuation of a magnetic field in magnetic resonance imaging (MRI). One method includes continuously obtaining respiration signal of a subject in a magnetic field; amplifying electrical signal corresponding to the obtained respiration signal according to a set of amplification parameters; and driving the amplified electrical signal into a solenoid coil to generate a feedback magnetic field to compensate respiration-induced fluctuation of the magnetic field in real-time.

### PRIORITY AND RELATED APPLICATION

This application claims priority to U.S. Provisional Application No. 63/635,144, filed Apr. 17, 2024, which is incorporated by reference in its entirety.

### TECHNICAL FIELD

This disclosure relates to magnetic resonance imaging (MRI), particularly for compensation of respiration induced B0 fluctuation in a real-time mode.

### BACKGROUND

Magnetic resonance imaging (MRI) is a widely deployed medical imaging in biomedical research and in clinics. Respiration-induced B0 fluctuations are a major source of confound in MRI and functional MRI (fMRI), wherein B0 field is a main static magnetic field in MRI. There are some issues, problems, and/or challenges associated with achieving real-time compensation of the respiration induced magnetic field fluctuation. For example, some approaches require sophisticated feedback control system.

The present disclosure describes various embodiments for providing respiration-feedback and correction with the respiration signal and a solenoid coil, addressing at least one of the issues, problems, and/or challenges discussed above, providing real-time correction of respiration-induced fluctuation of the B0 field in MRI, enhancing MRI image quantity and improving the technical field of MRI.

### SUMMARY

The present disclosure relates to methods, devices, and systems for enhancement of magnetic resonance imaging (MRI) by providing respiration-feedback and correction in real time.

The present disclosure describes a method for compensating respiration induced fluctuation of a magnetic field in magnetic resonance imaging (MRI), the method comprising: continuously obtaining respiration signal of a subject in a magnetic field; amplifying electrical signal corresponding to the obtained respiration signal according to a set of amplification parameters; and driving the amplified electrical signal into a solenoid coil to generate a feedback magnetic field to compensate respiration-induced fluctuation of the magnetic field in real-time.

The present disclosure describes a non-transitory computer readable storage medium storing computer readable instructions, wherein, the computer readable instructions, when executed by a processor, are configured to cause the processor to perform: continuously obtaining respiration signal of a subject in a magnetic field; amplifying electrical signal corresponding to the obtained respiration signal according to a set of amplification parameters; and driving the amplified electrical signal into a solenoid coil to generate a feedback magnetic field to compensate respiration-induced fluctuation of the magnetic field in real-time.

The present disclosure also describes an apparatus for compensating respiration induced fluctuation of a magnetic field in magnetic resonance imaging (MRI), the apparatus comprising: a memory storing instructions; and a processor in communication with the memory, wherein, when the processor executes the instructions, the processor is configured to cause the apparatus to perform: continuously obtaining respiration signal of a subject in a magnetic field; amplifying electrical signal corresponding to the obtained respiration signal according to a set of amplification parameters; and driving the amplified electrical signal into a solenoid coil to generate a feedback magnetic field to compensate respiration-induced fluctuation of the magnetic field in real-time.

The present disclosure also describes an apparatus including electric circuitry configured to implement any of the above methods.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The system, device, product, and/or method described below may be better understood with reference to the following drawings and description of non-limiting and non-exhaustive embodiments. The components in the drawings are not necessarily to scale. Emphasis instead is placed upon illustrating the principles of the present disclosure.

FIG. 1 shows a flow diagram of an embodiment of various methods in the present disclosure.

FIG. 2 shows a computer system that may be used to implement various components in an apparatus/device or various steps in a method described in the present disclosure.

FIG. 3A shows a diagram of an exemplary embodiment in the present disclosure.

FIG. 3B shows a diagram of an exemplary embodiment for a bias-correction circuit in the present disclosure.

FIG. 3C shows a diagram of one view of a solenoid coil relative to a magnetic resonance imaging (MRI) magnet in the present disclosure.

FIG. 3D shows a diagram of another view of a solenoid coil relative to a magnetic resonance imaging (MRI) magnet in the present disclosure.

FIG. 4 shows a diagram of an MRI device in the present disclosure.

FIG. 5A shows an MRI pulse sequence diagram of an exemplary embodiment in the present disclosure.

FIG. 5B shows a diagram of a rotation of spiral discs in k-space in the present disclosure.

FIG. 6 shows a magnitude image and a fieldmap acquired from 3D field-mapping sequence of an exemplary embodiment in the present disclosure.

FIG. 7A shows a plot of respiration signal and measured B0 field of an exemplary embodiment in the present disclosure.

FIG. 7B shows a plot of respiration signal and measured B0 field of another exemplary embodiment in the present disclosure.

FIG. 7C shows a plot of respiration signal and measured B0 field of another exemplary embodiment in the present disclosure.

FIG. 8A shows a plot of respiration signal and measured B0 field of another exemplary embodiment in the present disclosure.

FIG. 8B shows a plot of respiration signal and measured B0 field of another exemplary embodiment in the present disclosure.

### DETAILED DESCRIPTION

The disclosed systems, devices, and methods will now be described in detail hereinafter with reference to the accompanied drawings that form a part of the present application and show, by way of illustration, examples of specific embodiments. The described systems and methods may, however, be embodied in a variety of different forms and, therefore, the claimed subject matter covered by this disclosure is intended to be construed as not being limited to any of the embodiments. This disclosure may be embodied as methods, devices, components, or systems. Accordingly, embodiments of the disclosed system and methods may, for example, take the form of hardware, software, firmware or any combination thereof.

Throughout the specification and claims, terms may have nuanced meanings suggested or implied in context beyond an explicitly stated meaning. Likewise, the phrase “in one embodiment” or “in some embodiments” as used herein does not necessarily refer to the same embodiment and the phrase “in another embodiment” or “in other embodiments” as used herein does not necessarily refer to a different embodiment. It is intended, for example, that claimed subject matter may include combinations of exemplary embodiments in whole or in part. Moreover, the phrase “in one implementation”, “in another implementation”, “in some implementations”, or “in some other implementations” as used herein does not necessarily refer to the same implementation(s) or different implementation(s). It is intended, for example, that claimed subject matter may include combinations of the disclosed features from the implementations in whole or in part.

In general, terminology may be understood at least in part from usage in context. For example, terms, such as “and”, “or”, or “and/or,” as used herein may include a variety of meanings that may depend at least in part upon the context in which such terms are used. In addition, the term “one or more” or “at least one” as used herein, depending at least in part upon context, may be used to describe any feature, structure, or characteristic in a singular sense or may be used to describe combinations of features, structures or characteristics in a plural sense. Similarly, terms, such as “a”, “an”, or “the”, again, may be understood to convey a singular usage or to convey a plural usage, depending at least in part upon context. In addition, the term “based on” or “determined by” may be understood as not necessarily intended to convey an exclusive set of factors and may, instead, allow for existence of additional factors not necessarily expressly described, again, depending at least in part on context.

The present disclosure describes various embodiments for compensating respiration induced fluctuation of a magnetic field in magnetic resonance imaging (MRI) and/or functional MRI (fMRI).

Respiration-induced B0 fluctuations are a major source of confound in MRI/fMRI. In some implementations, real-time correction of respiration-induced fluctuation of B0 field in MRI has been developed using specially designed sensors and real-time system, which may require sophisticated feedback control system. In some implementations, respiration-induced B0 (the main static magnetic field) fluctuations are a major source of distortion in fMRI imaging; and this problem is known and gets worse at higher magnetic field (e.g., 7T).

Various embodiments in the present disclosure describe simple methods of respiration-feedback and correction using the respiration signal from the scanner and a solenoid coil located at the back of the magnet.

In various embodiments, a method may include a portion or all of the following: extracting the respiration cycle from the MRI console (respiration data is already collected during MRI); the respiratory waveform in the captured monitor screen is sampled in a Python script and converted to an audio signal; the audio signal is amplified and fed into the solenoid; solenoid induces a feedback B0 field that compensates for distortion due to respiration; and/or unreleased python code developed in house.

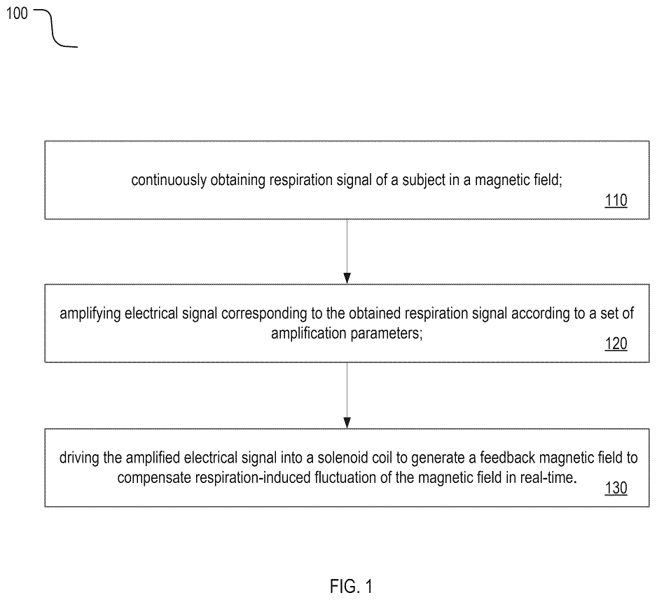

Referring to FIG. 1, the present disclosure describes various embodiments of a method 100 for compensating respiration induced fluctuation of a magnetic field in magnetic resonance imaging (MRI) and/or functional MRI (fMRI). The method may be performed by an electronic device comprising a portion or all of the following: one or more memories, one or more processors, one or more amplifiers, one or more circuitry, and one or more solenoid coils. The method includes a portion or all of the following: step 110, continuously obtaining respiration signal of a subject in a magnetic field; step 120, amplifying electrical signal corresponding to the obtained respiration signal according to a set of amplification parameters; and/or step 130, driving the amplified electrical signal into a solenoid coil to generate a feedback magnetic field to compensate respiration-induced fluctuation of the magnetic field in real-time.

In some implementations, the method further includes calibrating the set of amplification parameters by using a spin echo single voxel spectroscopy.

In some implementations, the set of amplification parameters comprises at least one of the following: an amplification ratio; and/or an amplification polarity.

In some implementations, the amplified electrical signal corresponding to the obtained respiration signal according to one or more types of general transfer functions that provides a mapping from the respiration signal to the average waveform of a frequency displacement.

In some implementations, the continuously obtaining the respiration signal of the subject comprises: continuously capturing video signal from an MRI console; and/or extracting the respiration signal of the subject from the captured video signal.

In some implementations, the MRI console is configured to receive the respiration signal of the subject from a respiration measuring device, which may include a respiration belt.

In some implementations, the method further includes, before amplifying electrical signal, performing signal processing on the electrical signal.

In some implementations, the signal processing comprises correcting a bias offset in the electrical signal by a bias-correction circuitry.

In some implementations, the solenoid coil is disposed near a magnet assembly that generating the magnetic field; and/or a central axis of the solenoid coil aligns with a central axis of the magnet assembly.

In some implementations, the solenoid coil comprises a plurality of turns of an electric wire.

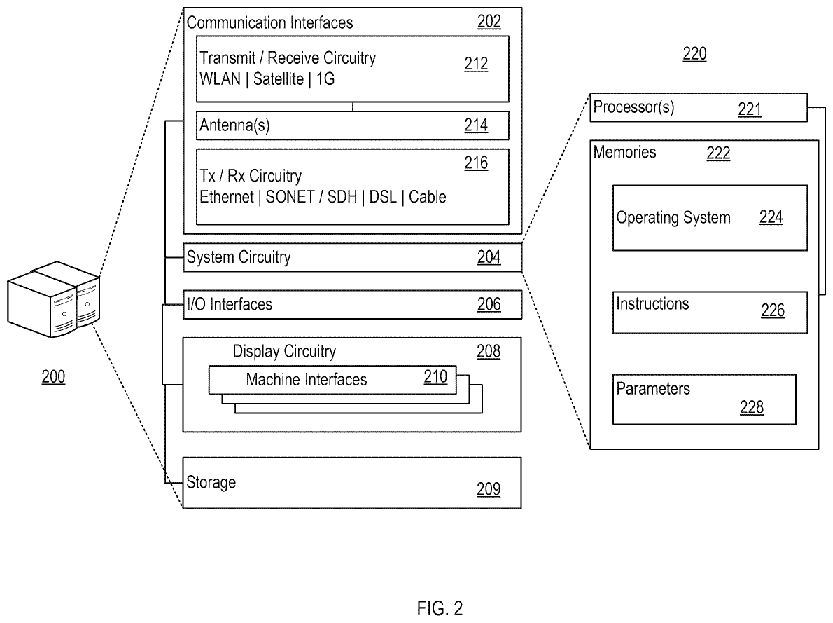

FIG. 2 shows an exemplary electronic device/apparatus, wherein any device, unit, or module in the present disclosure may include a portion or all of the components in the exemplary electronic device/apparatus. The electronic device/apparatus may include a computer system 200 for implementing one or more steps in various embodiments of the present disclosure. The computer system 200 may include communication interfaces 202, system circuitry 204, input/output (I/O) interfaces 206, storage 209, and display circuitry 208 that generates machine interfaces 210 locally or for remote display, e.g., in a web browser running on a local or remote machine. For one example, the computer system 200 may communicate with one or more instrument (e.g., an RF coil).

The machine interfaces 210 and the I/O interfaces 206 may include GUIs, touch sensitive displays, voice or facial recognition inputs, buttons, switches, speakers and other user interface elements. Additional examples of the I/O interfaces 206 include microphones, video and still image cameras, headset and microphone input/output jacks, Universal Serial Bus (USB) connectors, general purpose digital interface (GPIB), peripheral component interconnect (PCI), PCI extensions for instrumentation (PXI), memory card slots, and other types of inputs. The I/O interfaces 206 may further include magnetic or optical media interfaces (e.g., a CDROM or DVD drive), serial and parallel bus interfaces, and keyboard and mouse interfaces.

The communication interfaces 202 may include wireless transmitters and receivers (“transceivers”) 212 and any antennas 214 used by the transmitting and receiving circuitry of the transceivers 212. The transceivers 212 and antennas 214 may support Wi-Fi network communications, for instance, under any version of IEEE 802.11, e.g., 802.11n or 802.11ac. The communication interfaces 202 may also include wireline transceivers 216. The wireline transceivers 216 may provide physical layer interfaces for any of a wide range of communication protocols, such as any type of Ethernet, data over cable service interface specification (DOCSIS), digital subscriber line (DSL), Synchronous Optical Network (SONET), or other protocol.

The storage 209 may be used to store various initial, intermediate, or final data or model for implementing the embodiment for determining at least one reaction condition. These data corpus may alternatively be stored in a database 118. In one implementation, the storage 209 of the computer system 200 may be integral with a database. The storage 209 may be centralized or distributed, and may be local or remote to the computer system 200. For example, the storage 209 may be hosted remotely by a cloud computing service provider.

The system circuitry 204 may include hardware, software, firmware, or other circuitry in any combination. The system circuitry 204 may be implemented, for example, with one or more systems on a chip (SoC), application specific integrated circuits (ASIC), microprocessors, discrete analog and digital circuits, and other circuitry.

For example, at least some of the system circuitry 204 may be implemented as processing circuitry 220. The processing circuitry 220 may include one or more processors 221 and memories 222. The memories 222 stores, for example, control instructions 226, parameters 228, and/or an operating system 224. The control instructions 226, for example may include instructions for implementing one or more steps of various embodiments. In one implementation, the instruction processors 221 execute the control instructions 226 and the operating system 224 to carry out any desired functionality related to the embodiment.

Various embodiments/implementations may be performed by a portion or all of the components in the electronic device as shown in FIG. 2.

Some implementations include the respiration-feedback system using the respiration monitor signal available at the MRI console and a solenoid shim coil that has been developed for direct neuronal current mapping.

Some implementations achieve the goal(s) to compensate for the respiration induced B0 fluctuation in a real-time mode.

In some implementations, the respiration signal was extracted from the MRI console display using a real-time screen capture technique. The extracted respiration signal was adjusted and driven to the solenoid shim coil using a custom-built amplifier. Its function was checked by monitoring a resonance frequency at a single-voxel-selective MR spectroscopy (MRS) voxel located at an occipital lobe.

In some implementations, the B0 fluctuation was compensated by the captured respiration signal in real-time.

In some implementations, the respiration signal which is available at any MRI console can be used for real-time feedback with a video-capturing USB device to compensate for the respiration induced B0 fluctuation. This will make the respiration feedback system more easily implementable.

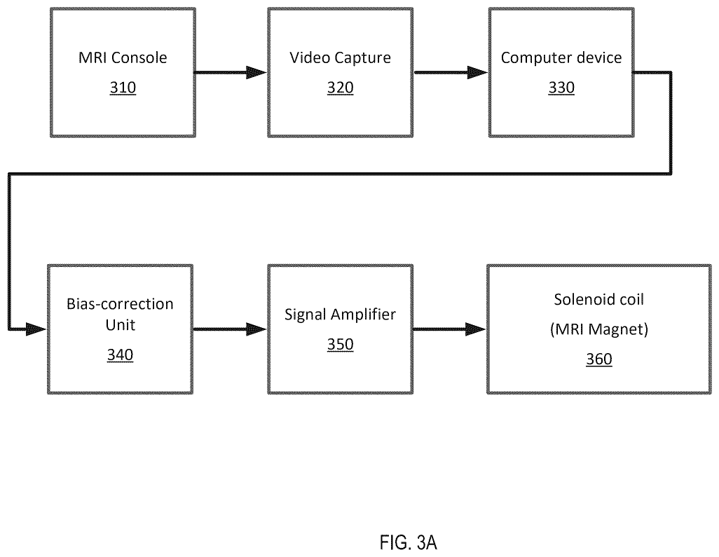

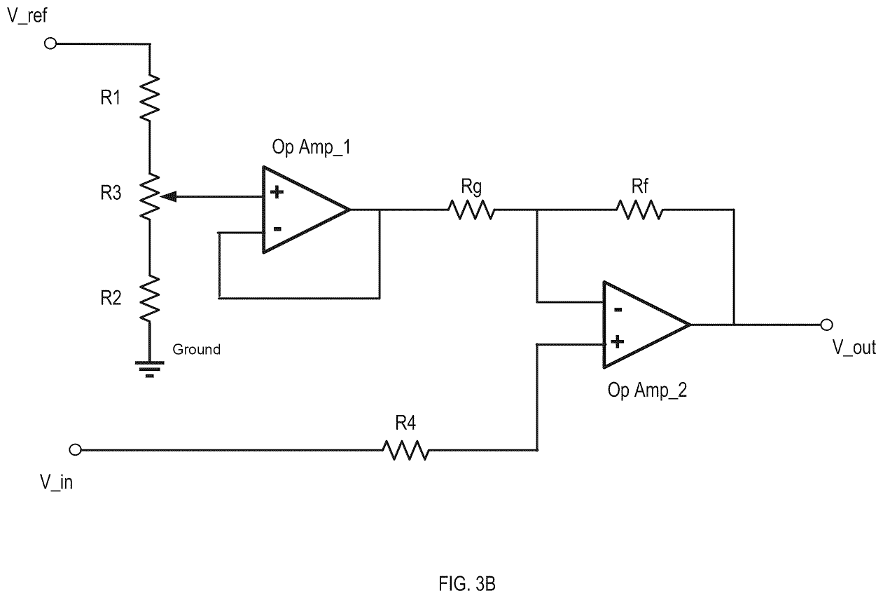

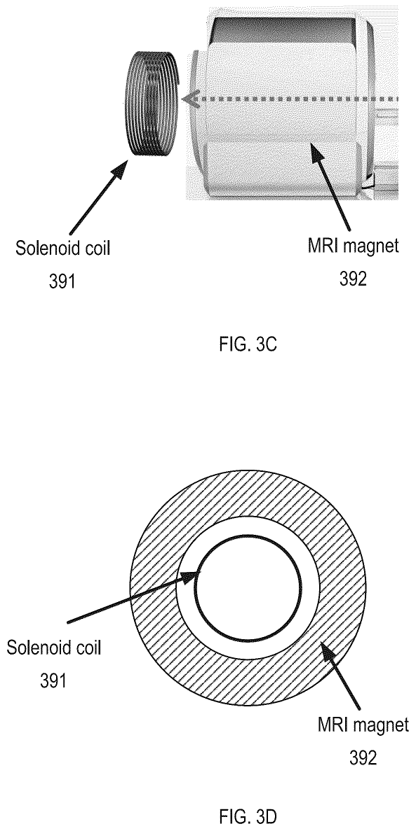

FIG. 3A shows a block diagram of the real-time respiratory feedback system. The monitor screen of an MRI console (310) is continuously captured using a video capture device 320 (for example by a video-capturing universal serial bus (USB) device). The video capture may be performed via a high-definition multimedia interface (HDMI) from the MRI console.

The captured video data may be read into a computer device 330. In some implementations, the computer device may be a stimuli personal computer (PC), which may be used to generate and present stimuli (e.g., visual, auditory) during MRI. The computer device may process the captured video data (including respiratory waveform) and output signal. In some implementations, the respiratory waveform in the captured monitor screen may be sampled in a Python script running on the computer device and converted to an audio signal. In some implementations, the computer device may include a direct current (DC) coupled sound card, and the audio signal may be generated and output by the DC-coupled sound card.

The audio signal may be transmitted to a bias-correction unit 340 to correct for its bias (or bias offset). In some implementations, the bias-correction unit 340 may include a bias-correction circuit board, and receive its input from the computer device via a 3.5 millimeter (mm) audio interface. FIG. 3B shows one exemplary circuit to be included in the bias-correction unit. For one non-limiting example, R1 may be about 6.7 kilo-Ohm (kΩ), R2 may be a potentiometer having a range between 0 and 2 kΩ, inclusive, R3 is about 2 kΩ, Rg is about 1 kΩ, Rf is about 2.2 kΩ, R4 is about 1 kΩ; V_ref is a reference voltage, V_in is an input voltage of the bias-correction unit, and V_out is an output of the correction unit; and/or Op Amp_1 is a first operational amplifier (a type of differential amplifier), and Op Amp_2 is a second operational amplifier (a type of differential amplifier). In one implementation, the operational amplifier may be a high-performance, monolithic, dual operational amplifier.

The output from the bias-correction unit 340 may be received by a signal amplifier 350, for example, via a coaxial cable. The signal amplifier may amplify the input signal to be fed into a solenoid coil in the MRI magnet 360. In some implementations, the signal amplifier may include an audio amplifier that drives a solenoid coil from a bias-corrected respiration signal.

The solenoid coil may be a multi-turn coil in an MRI magnet, for example, being a built in part of the MRI magnetic coil, or being an add-on part to the MRI magnetic coil. In a non-limiting example, referring to FIG. 3C and FIG. 3D, the solenoid coil 391 may be co-axial with the MRI magnet assembly 392, i.e., a central axis of the solenoid coil aligning with a central axis of the magnet assembly. As an add-on coil, the solenoid coil may be disposed on one side of the MRI magnetic assembly.

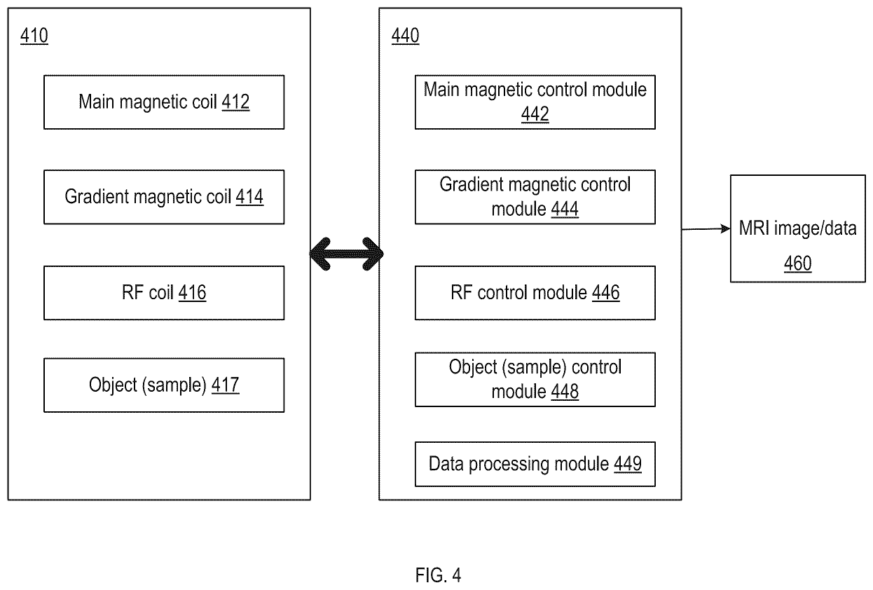

A schematic diagram of an exemplary MRI device that is used in various embodiments in the present disclosure is shown in FIG. 4. The MRI device may include a magnetic assembly unit 410 and a system controller 440. In some implementations, the magnetic assembly unit 410 may include a portion or all of the following: one or more main magnetic coil 412, one or more gradient magnetic coil 414, one or more RF coil 416, and/or an object (or sample) and its supporting device 417. The system controller 440 may include a portion or all of the following: a main magnetic control module 442 (communicating with and/or powering one or more main magnetic coil); a gradient magnetic control module 444 (communicating with and/or powering one or more gradient magnetic coil); an RF control module 446 (communicating with and/or powering one or more RF coil); an object (sample) control module (communicating with and/or powering one or more object (sample)'s supporting device; and/or a data processing module 449. In some implementations, the data processing module may process raw data acquired from the magnetic assembly unit, and/or process image data (e.g., for output or display MRI image/data 460).

In various embodiments, MRI imaging may be based on sensing the magnetization properties of atomic nuclei in an object (sample). Normally, proton magnetization within water of the object (sample) is randomly oriented. When a strong external magnetic field is applied, the proton magnetization may be aligned. This alignment (or magnetization) may be perturbed/excited by one or more RF pulse. When the perturbation of the magnetization relaxes, the nuclei may return to un-perturbed (or resting) alignment, during which the nuclei may emit a radio frequency energy. The emitted radio frequency energy, which may be referred as echo signal, may be detected and measured for generating MRI images of the object (sample).

In some implementations, repetition time (TR) may refer to the amount of time duration between successive RF pulse sequences applied to a slice of the sample. Echo time (TE) may refer to the amount of time duration between the RF pulse and the echo signal.

In some implementations, signal processing (e.g., Fourier transformation) may be used to construct the MRI image. In some implementations, Fourier transformation may convert frequency information contained in the echo signal for locations in image plan to intensity level of certain pixels. At least two different relaxation time may be used to characterize the signal: T1 refers to a longitudinal relaxation time, and T2 refers to a transverse relaxation time. In some implementations, T1 is determined by a rate at which the excited nuclei (e.g., proton) return to resting state (i.e., equilibrium), and corresponds to the time duration for protons to realign with the strong external magnetic field. In some implementations, T2 is determined by a rate at which excited protons go out of phase with each other and corresponds to the time duration for protons to lose phase coherence. In some implementations, MRI images may include T1-weighted MRI image, T2-weighted MRI image.

In some implementations, respiration signal may be obtained from a respiration measuring device. For a non-limiting example, the respiration measuring device may include a respiration belt and/or a respiratory cushion. In some implementations, the respiratory cushion may be with a pressure hose. For a breathing style being thoracic, the respiratory cushion may be placed on the chest of the subject; and/or for a breathing style being abdomen, the respiratory cushion may be placed on the abdomen of the subject.

In some implementations for system components configured in a control room, a video capturing USB device may receive MRI console video signal through a HDMI cable and send the captured video to a stimuli PC through an USB cable. The captured video signal is displayed on a monitor; a bias (or bias offset) of an audio output from the stimuli PC is corrected at a circuit board and may be monitored on an oscilloscope. A custom-made audio amplifier may amply the bias-corrected signal, and the output from the audio amplifier is used to drive the solenoid coil.

In one exemplary embodiment, the respiration waveform displayed on the MRI console was captured into a stimuli computer configured for fMRI through a video capturing USB device. A vendor-supplied pneumatic respiration device was used at a 3T whole body MRI scanner. The respiration signal was extracted from the captured console using a CV2 library in a Python script. The respiration signal was then fed into a sound card that was modified into a DC-coupled circuit by shunting a capacitor on the output channel. The DC-coupled respiration signal was first corrected for a bias at a custom-built bias correction circuit board using operational amplifiers (OP amplifiers), e.g., MC1458, and then it was driven into a solenoid coil through an audio amplifier with an OP amplifier, e.g., OPA548. The solenoid coil was constructed using a copper wire (e.g., 10 turns and diameter of 49 cm) and it was attached to a plastic stand. It was located 10 cm distance from the rear cover of the 3T magnet.

In some implementations, the B0 magnetic field map was measured at full inhale and exhale breath-hold using a 3-D fast (scan time=9 s) field-mapping sequence that was developed by extending the RSD ultra-short echo time (UTE) sequence to acquire two echoes at echo times=0.07 and 4.8 ms with a 3.4 mm isotropic voxel size, TR=9.8 ms, and flip angle=7°.

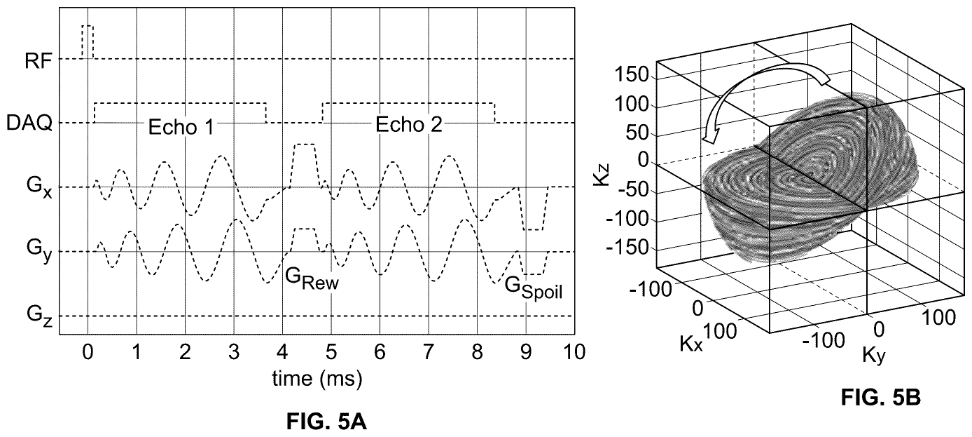

FIG. 5A shows an MRI pulse sequence diagram of one TR, and FIG. 5B shows a rotation of spiral discs to fill a 3D k-space. The echo time difference was 4.73 ms. The spiral readout of the first echo was rewound using the rewind gradient pulses (G_{Rew}) and the spiral readout of the second echo was spoiled using the spoil gradient pulses (G_{Spoil}).

In some implementations, the field maps were obtained at full inhale and exhale breath-hold conditions to calibrate the polarity, amplitude, and delay of the correction current to the solenoid. The feedback system was tested on two subjects in natural breathing experiments in which the frequency of a water spectral peak was monitored together with a respiration signal using a single-voxel-selective MR spectroscopy (MRS) sequence at a right visual cortex region (a cubic voxel with 15 mm side, TR/TE= 250/33 ms, flip angle=40°, 128 measurements, and vector size=256). Frequency values at each TR were calculated using the FID-A toolbox. The effect of feedback was measured by standard deviation of the B0 field fluctuation and by a correlation coefficient between the respiratory and the B0 field fluctuation.

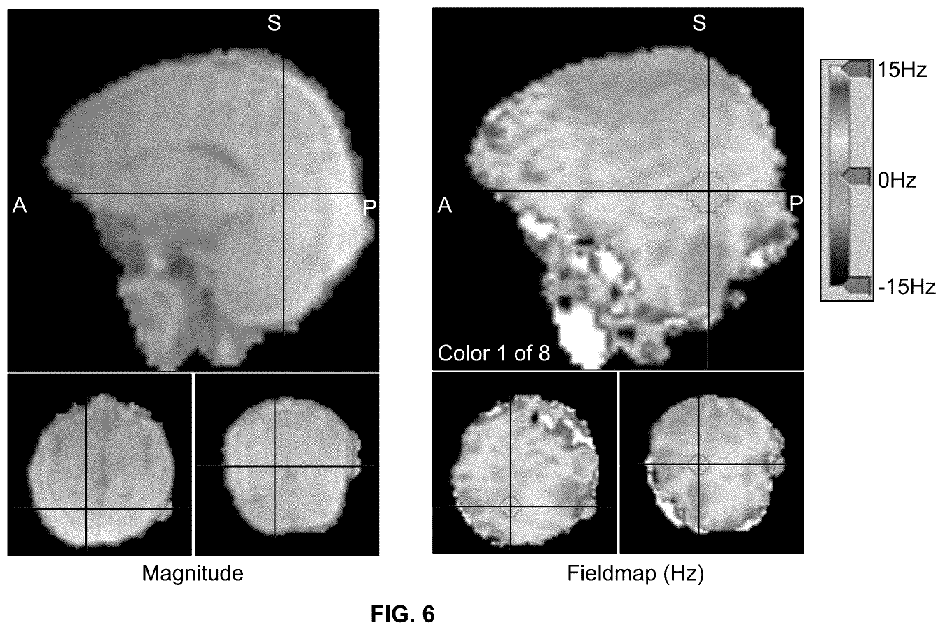

In some implementations, there was no detectable lag from the respiration signal displayed on the scanner's console and the output waveform from the audio amplifier. The solenoid coil was noticed to sway a few millimeters following the driving respiratory waveform. Measurement results from one representative subject (age=24, weight=86 kg, height=182 cm) is shown here. The field maps acquired at full inhale and exhale conditions resulted in 3.0 Hz difference in frequency at the occipital lobe region (FIG. 6).

FIG. 6 shows a magnitude image and a fieldmap acquired from 3D field-mapping sequence. The fieldmap was a difference between the fieldmaps acquired at inhale and exhale breath-hold states. The fieldmap difference in the circled region of a visual cortex was 3.0 Hz using the echo time difference of 4.73 ms. In the field map, there was a gradient or slope in the superior-inferior as well as in the anterior-to-posterior directions.

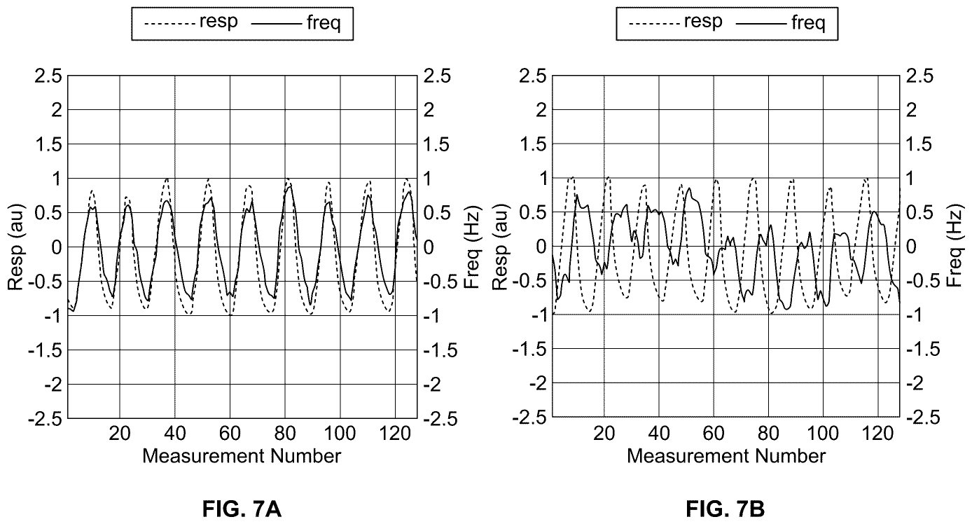

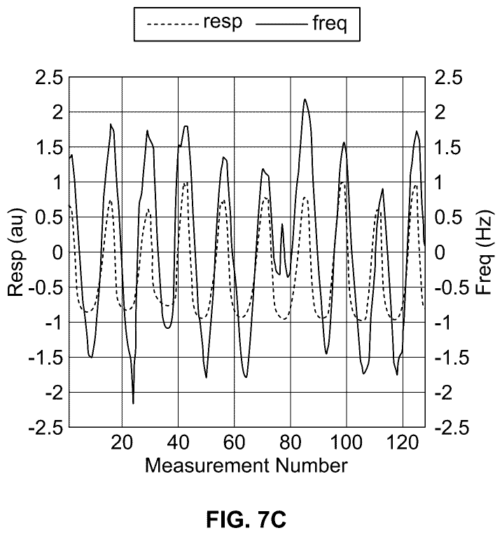

In some implementations, the B0 or frequency fluctuation during natural breathing is shown in FIG. 7A in the absence and presence of respiration feedback. The respiration feedback effect with the non-inverting polarity (in FIG. 7B) resulted in decreased standard deviation and correlation, while that with the inverting polarity (in FIG. 7C) resulted in increased standard deviation and higher correlation coefficient from the non-inverted feedback.

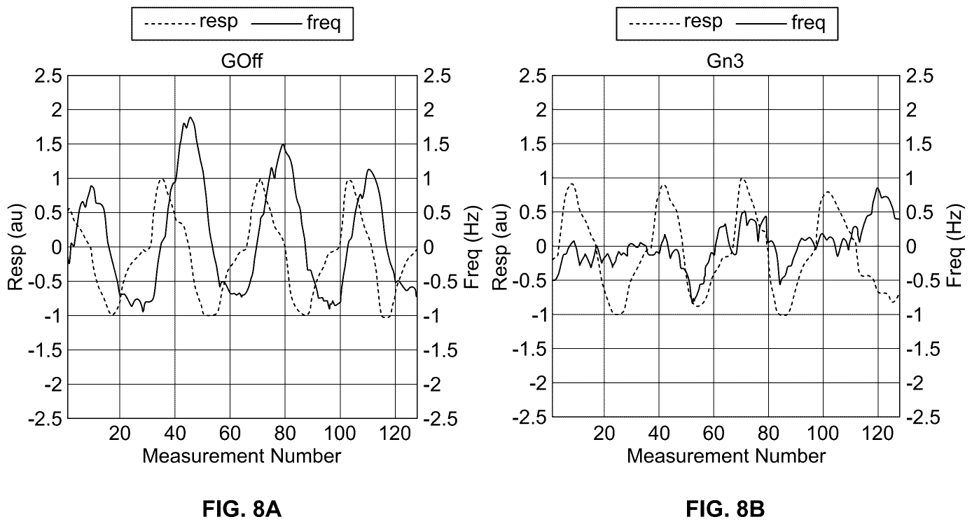

FIGS. 8A-8B show another example for plots of respiration signal and measured B0 field in two different conditions of the feedback control: with the amplifier off (in FIG. 8A), and with the feedback of an inverted respiration signal (in FIG. 8B).

In some implementations, the calibration of the feedback variables such as the gain and delay time may be implemented as an effective calibration method. The solenoid-coil-induced field is sloped in the solenoid axis or the Z direction, and this may limit the field correction area. However, the solenoid-coil-induced field may be calibrated at a small region as in MR spectroscopy. In some implementations, a more general correction can be obtained for real-time control of the existing shim coils in the MRI system.

The present disclosure describes a real-time feedback system of respiration with minimum additional components and demonstrates its effectiveness in compensating the respiration induced B0 field at a single voxel region. In some implementations, the present disclosure also describes a fast B0 field mapping sequence using a 3D UTE sequence that acquired the field maps during a short period of breath hold.

Various embodiments in the present disclosure may include an apparatus for compensating respiration induced fluctuation of a magnetic field in magnetic resonance imaging (MRI). The apparatus may include a memory storing instructions; and a processor in communication with the memory, wherein, when the processor executes the instructions, the processor is configured to cause the apparatus to perform: continuously obtaining respiration signal of a subject in a magnetic field; amplifying electrical signal corresponding to the obtained respiration signal according to a set of amplification parameters; and driving the amplified electrical signal into a solenoid coil to generate a feedback magnetic field to compensate respiration-induced fluctuation of the magnetic field in real-time.

In some implementations, the apparatus may include the solenoid coil.

In some implementations, when the processor executes the instructions, the processor is configured to further cause the apparatus to perform: calibrating the set of amplification parameters by using a spin echo single voxel spectroscopy.

In some implementations, the set of amplification parameters comprises at least one of the following: an amplification ratio; an amplification polarity; and/or a phase delay.

In some implementations, the continuously obtaining the respiration signal of the subject comprises: continuously capturing video signal from an MRI console; and/or extracting the respiration signal of the subject from the captured video signal.

In some implementations, the MRI console is configured to receive the respiration signal of the subject from a respiration measuring device.

In some implementations, when the processor executes the instructions, the processor is configured to further cause the apparatus to perform: before amplifying electrical signal, performing signal processing on the electrical signal.

In some implementations, the signal processing comprises correcting a bias offset in the electrical signal by a bias-correction circuitry.

In some implementations, the solenoid coil is disposed near a magnet assembly that generating the magnetic field; and/or a central axis of the solenoid coil aligns with a central axis of the magnet assembly.

In some implementations, the solenoid coil comprises a plurality of turns of an electric wire.

Various embodiments in the present disclosure may include a non-transitory computer readable storage medium storing computer readable instructions, wherein, the computer readable instructions, when executed by a processor, are configured to cause the processor to perform: continuously obtaining respiration signal of a subject in a magnetic field; amplifying electrical signal corresponding to the obtained respiration signal according to a set of amplification parameters; and driving the amplified electrical signal into a solenoid coil to generate a feedback magnetic field to compensate respiration-induced fluctuation of the magnetic field in real-time.

In some implementations, the computer readable instructions, when executed by the processor, are configured to cause the processor to perform: calibrating the set of amplification parameters by using a spin echo single voxel spectroscopy.

In some implementations, the set of amplification parameters comprises at least one of the following: an amplification ratio; an amplification polarity; and/or a phase delay.

In some implementations, the continuously obtaining the respiration signal of the subject comprises: continuously capturing video signal from an MRI console; and extracting the respiration signal of the subject from the captured video signal.

In some implementations, the MRI console is configured to receive the respiration signal of the subject from a respiration measuring device.

In some implementations, the computer readable instructions, when executed by the processor, are configured to cause the processor to perform: before amplifying electrical signal, performing signal processing on the electrical signal.

In some implementations, the signal processing comprises correcting a bias offset in the electrical signal by a bias-correction circuitry.

In some implementations, the solenoid coil is disposed near a magnet assembly that generating the magnetic field; and a central axis of the solenoid coil aligns with a central axis of the magnet assembly.

In some implementations, the solenoid coil comprises a plurality of turns of an electric wire.

In various embodiments and/or implementations of this disclosure, any step and/or any operation and/or implementation may be combined or arranged in any amount or order, as desired. Two or more of the steps and/or operations and/or implementations may be performed in parallel. Embodiments and implementations in the disclosure may be used separately or combined in any order. Further, each of the methods (or embodiments), an encoder, and a decoder may be implemented by processing circuitry (e.g., one or more processors or one or more integrated circuits).

Various embodiments provide real-time correction of respiration-induced fluctuation of the B0 field in MRI, providing a solution for respiration-induced B0 (which is the main static magnetic field) fluctuations as a major source of distortion in MRI/fMRI imaging.

The methods, devices, processing, and logic described above and below may be implemented in many different ways and in many different combinations of hardware and software. For example, all or parts of the implementations may be circuitry that includes an instruction processor, such as a Graphics Processing Unit (GPU), Central Processing Unit (CPU), microcontroller, or a microprocessor; an Application Specific Integrated Circuit (ASIC), Programmable Logic Device (PLD), or Field Programmable Gate Array (FPGA); or circuitry that includes discrete logic or other circuit components, including analog circuit components, digital circuit components or both; or any combination thereof. The circuitry may include discrete interconnected hardware components and/or may be combined on a single integrated circuit die, distributed among multiple integrated circuit dies, or implemented in a Multiple Chip Module (MCM) of multiple integrated circuit dies in a common package, as examples.

The circuitry may further include or access instructions for execution by the circuitry. The instructions may be embodied as a signal and/or data stream and/or may be stored in a tangible storage medium that is other than a transitory signal (i.e., non-transitory signal), such as a flash memory, a Random Access Memory (RAM), a Read Only Memory (ROM), an Erasable Programmable Read Only Memory (EPROM); or on a magnetic or optical disc, such as a Compact Disc Read Only Memory (CDROM), Hard Disk Drive (HDD), or other magnetic or optical disk; or in or on another machine-readable medium. A product, such as a computer program product, may particularly include a non-transitory storage medium and instructions stored in or on the medium, and the instructions when executed by the circuitry in a device may cause the device to implement any of the processing described above or illustrated in the drawings.

The implementations may be distributed as circuitry, e.g., hardware, and/or a combination of hardware and software among multiple system components, such as among multiple processors and memories, optionally including multiple distributed processing systems. Parameters, databases, and other data structures may be separately stored and managed, may be incorporated into a single memory or database, may be logically and physically organized in many different ways, and may be implemented in many different ways, including as data structures such as linked lists, hash tables, arrays, records, objects, or implicit storage mechanisms. Programs may be parts (e.g., subroutines) of a single program, separate programs, distributed across several memories and processors, or implemented in many different ways, such as in a library, such as a shared library (e.g., a Dynamic Link Library (DLL)). The DLL, for example, may store instructions that perform any of the processing described above or illustrated in the drawings, when executed by the circuitry.

In various embodiments in the present disclosure, a module may refer to a software module, a hardware module, or a combination thereof. A software module may include a computer program or part of the computer program that has a predefined function and works together with other related parts to achieve a predefined goal, such as those functions described in this disclosure. A hardware module may be implemented using processing circuitry and/or memory configured to perform the functions described in this disclosure. Each module can be implemented using one or more processors (or processors and memory). Likewise, a processor (or processors and memory) can be used to implement one or more modules. Moreover, each module can be part of an overall module that includes the functionalities of the module. The description here also applies to the term module and other equivalent terms.

While the particular disclosure has been described with reference to illustrative embodiments, this description is not meant to be limiting. Various modifications of the illustrative embodiments and additional embodiments of the disclosure will be apparent to one of ordinary skill in the art from this description. Those skilled in the art will readily recognize that these and various other modifications can be made to the exemplary embodiments, illustrated and described herein, without departing from the spirit and scope of the present disclosure. It is therefore contemplated that the appended claims will cover any such modifications and alternate embodiments. Certain proportions within the illustrations may be exaggerated, while other proportions may be minimized. Accordingly, the disclosure and the figures are to be regarded as illustrative rather than restrictive.

What is claimed is:

1. A method for compensating respiration induced fluctuation of a magnetic field in magnetic resonance imaging (MRI), the method comprising:
continuously obtaining respiration signal of a subject in a magnetic field;
amplifying electrical signal corresponding to the obtained respiration signal according to a set of amplification parameters; and
driving the amplified electrical signal into a solenoid coil to generate a feedback magnetic field to compensate respiration-induced fluctuation of the magnetic field in real-time.

2. The method according to claim 1, further comprising:
calibrating the set of amplification parameters by using a spin echo single voxel spectroscopy.

3. The method according to claim 1, wherein:
the set of amplification parameters comprises at least one of the following:
an amplification ratio;
an amplification polarity; or
a phase delay.

4. The method according to claim 1, wherein the continuously obtaining the respiration signal of the subject comprises:
continuously capturing video signal from an MRI console; and
extracting the respiration signal of the subject from the captured video signal.

5. The method according to claim 4, wherein:
the MRI console is configured to receive the respiration signal of the subject from a respiration measuring device.

6. The method according to claim 1, further comprising:
before amplifying electrical signal, performing signal processing on the electrical signal.

7. The method according to claim 1, wherein:
the signal processing comprises correcting a bias offset in the electrical signal by a bias-correction circuitry.

8. The method according to claim 1, wherein:
the solenoid coil is disposed near a magnet assembly that generating the magnetic field; and
a central axis of the solenoid coil aligns with a central axis of the magnet assembly.

9. The method according to claim 1, wherein:
the solenoid coil comprises a plurality of turns of an electric wire.

10. An apparatus for compensating respiration induced fluctuation of a magnetic field in magnetic resonance imaging (MRI), the apparatus comprising:
a memory storing instructions; and
a processor in communication with the memory, wherein, when the processor executes the instructions, the processor is configured to cause the apparatus to perform:
continuously obtaining respiration signal of a subject in a magnetic field;
amplifying electrical signal corresponding to the obtained respiration signal according to a set of amplification parameters; and
driving the amplified electrical signal into a solenoid coil to generate a feedback magnetic field to compensate respiration-induced fluctuation of the magnetic field in real-time.

11. The apparatus according to claim 10, wherein, when the processor executes the instructions, the processor is configured to further cause the apparatus to perform:
calibrating the set of amplification parameters by using a spin echo single voxel spectroscopy.

12. The apparatus according to claim 10, wherein:
the set of amplification parameters comprises at least one of the following:
an amplification ratio;
an amplification polarity; or
a phase delay.

13. The apparatus according to claim 10, wherein the continuously obtaining the respiration signal of the subject comprises:
continuously capturing video signal from an MRI console; and
extracting the respiration signal of the subject from the captured video signal.

14. The apparatus according to claim 13, wherein:
the MRI console is configured to receive the respiration signal of the subject from a respiration measuring device.

15. The apparatus according to claim 10, wherein, when the processor executes the instructions, the processor is configured to further cause the apparatus to perform:
before amplifying electrical signal, performing signal processing on the electrical signal.

16. A non-transitory computer readable storage medium storing computer readable instructions, wherein, the computer readable instructions, when executed by a processor, are configured to cause the processor to perform:
continuously obtaining respiration signal of a subject in a magnetic field;
amplifying electrical signal corresponding to the obtained respiration signal according to a set of amplification parameters; and
driving the amplified electrical signal into a solenoid coil to generate a feedback magnetic field to compensate respiration-induced fluctuation of the magnetic field in real-time.

17. The non-transitory computer readable storage medium according to claim 16, wherein, the computer readable instructions, when executed by the processor, are configured to cause the processor to perform:
calibrating the set of amplification parameters by using a spin echo single voxel spectroscopy.

18. The non-transitory computer readable storage medium according to claim 16, wherein:
the set of amplification parameters comprises at least one of the following:
an amplification ratio;
an amplification polarity; or
a phase delay.

19. The non-transitory computer readable storage medium according to claim 16, wherein the continuously obtaining the respiration signal of the subject comprises:
continuously capturing video signal from an MRI console; and
extracting the respiration signal of the subject from the captured video signal.

20. The non-transitory computer readable storage medium according to claim 19, wherein:
the MRI console is configured to receive the respiration signal of the subject from a respiration measuring device.
